(19) United States
(12) Patent Application Publication
(10) Pub. No.: US 2014/0341817 A1
(43) Pub. Date: Nov. 20, 2014

(54) PROPELLENT-CONTAINING COSMETIC COMPOSITION

(71) Applicant: Henkel AG & Co. KGaA, Dusseldorf (DE)

(72) Inventors: Burkhard Mueller, Duesseldorf (DE); Anja Thammasiri, Klein Nordende (DE)

(21) Appl. No.: 14/361,597

(22) PCT Filed: Nov. 9, 2012
(86) PCT No.: PCT/EP2012/072244

§ 371 Date: May 29, 2014

(30) Foreign Application Priority Data
Dec. 9, 2011 (DE) 10 2011 088 148.4

(51) Int. Cl. A61K 8/04; A61Q 5/06; A61K 8/86

(52) U.S. Cl. CPC A61K 8/046; A61K 8/86; A61Q 5/06
USPC 424/47

(57) ABSTRACT

Cosmetic agents for the cosmetic treatment of keratin fibres contain in a cosmetic carrier, i) at least one polymer and; ii) at least one compound of the formula R¹—X¹—(O—CH₂CH₂)n—OH (I), comprising at most 20 ethylene oxide units iii) at least one linear or branched compound of the formula:

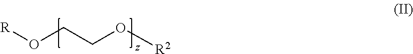

with at least 30 ethylene oxide units; and iv) at least one propellant. The cosmetic agents effect a foam which permits styling with a high degree of hold, high flexibility and good care and does not weigh down the hair.

### CROSS REFERENCE TO RELATED APPLICATION

The present disclosure is a U.S. National Stage entry under 35 U.S.C. §371 based on International Application No. PCT/EP2012/072244, filed Nov. 9, 2012, which claims priority to German Patent Application No. DE 10 2011 088 148.4 filed on Dec. 9, 2011, which is incorporated herein in its entirety by reference.

### TECHNICAL FIELD

The technical field relates to cosmetic agents with propellant.

### BACKGROUND

Healthy hair and an attractive-looking hairstyle are generally regarded today as an essential element of a well-groomed exterior. In general, dull hair or thin hair is perceived as an esthetic flaw. If the hair is exposed to environmental influences, such as, e.g., UV radiation, mechanical stress or free radicals, the hair structure can be negatively affected. This has an impact on the external appearance. In the case of so-called split ends, for example, the tips of the hair have visibly degenerated in their hair structure, leaving the hair looking sickly and unhealthy. People associate natural gloss and fullness of the hair with an ideal of beauty, which they wish to claim for themselves. Furthermore, they wish to bring the hair into a shape that pleases them (hairstyle) and to fix this shape. As auxiliary agents for this, cosmetic agents are used which protect the hair or modify the hair in its properties in such a way that the appearance of the hair approximates to the desired ideal of beauty.

Appropriate agents for temporary shaping usually contain fixing polymers as a shaping component. Preparations that contain a dissolved or dispersed polymer can be applied onto the hair by means of propellants or by a pump mechanism. In particular, hair gels and hair waxes are not generally applied onto the hair directly but are distributed in the hair by means of a comb or the hands.

Application as a cosmetic foamed by means of propellant gas has gained broad acceptance, since foams can be distributed on the hair simply and uniformly. However, the foams must comply with a particular requirement profile so that the application can be carried out optimally and the action of the polymers on the hair can develop appropriately. The foams must form rapidly, remain stable for a short time and break down on the hair so that no permanent covering of foam is formed. The foam must not weigh the hair down; otherwise the style that has been applied will drop out and thus be lost.

The most important property of an agent for the temporary shaping of keratinic fibers, also referred to below as a styling agent, consists in giving the treated fibers the strongest possible hold in the shape that has been produced. If the keratinic fibers are human hair, this is also described as a strong styling hold or a high degree of hold of the styling agent. The styling hold is substantially determined by the nature and quantity of the fixing polymer that is used, although the further constituents of the styling agent and the type of application can also have an influence.

In addition to having a high degree of hold, styling agents also have to satisfy a whole series of further requirements. Mention can be made in particular of moisture resistance, low tackiness and a balanced conditioning effect. The polymers ideally form a polymer film when applied to the hair, which on the one hand provides the hairstyle with strong hold but on the other is sufficiently flexible not to break under stress. If the polymer film is too brittle, so-called film flakes are formed, i.e. residues which detach when the hair is moved and give the impression that the user of the corresponding styling agent has dandruff.

### SUMMARY

Accordingly, cosmetic agents with propellant gas, which are distinguished by a high degree of hold, are provided herein. In an exemplary embodiment, the agents simultaneously exhibit flexible hold together with a high care performance. The formation of film flakes—in particular the formation of corresponding residues during application, brushing out and drying of the keratin-containing fibers—should be avoided. In an exemplary embodiment, the agents take the form of a foam and the foam forms rapidly and only begins to break down significantly upon application on the hair. The foam is provided that does not weigh down the hair. It has now been found that this is achieved by a combination of two specific nonionic surfactants.

### DETAILED DESCRIPTION

In accordance with an exemplary embodiment, a cosmetic agent for the cosmetic treatment of keratinic fibers, in particular human hair, contains in a cosmetic carrier:
i) at least one polymer;
ii) at least one compound of formula (I), comprising no more than 20 ethylene oxide units

R¹—X¹—(O—CH₂CH₂)ₙ—OH (I)
where
R¹ denotes a linear or branched, saturated or unsaturated hydrocarbon residue with 8 to 20 carbon atoms,
X¹ denotes a direct bond or a carbonyl group,
n denotes an integer from 1 to 20;
iii) at least one linear or branched compound of formula (II) with at least 30 ethylene oxide units

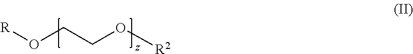

where
R² denotes a linear or branched (C₇ to C₂₁) alkylcarbonyl group, a linear or branched (C₇ to C₂₁) alkenylcarbonyl group or denotes a linear or branched, saturated or unsaturated (C₈ to C₂₂) hydrocarbon residue,
R denotes a hydrogen atom or a group of formula (III)

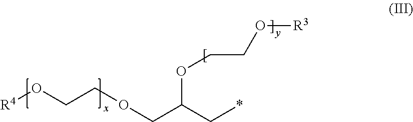

in which x and y independently of one another denote an integer from 0 to 80, R³ and R⁴ independently of one another denote a hydrogen atom or a linear or branched, saturated or unsaturated (C₇ to C₂₁) alkylcarbonyl group,
z denotes an integer from 0 to 80,
with the provisos that
   if R is a group of formula (III), at least one group from R², R³, R⁴ denotes a linear or branched (C₇ to C₂₁) alkylcarbonyl group or a linear or branched (C₇ to C₂₁) alkenylcarbonyl group and the sum of x+y+z is at least 30,
   if R denotes a hydrogen atom, z signifies an integer from 30 to 80,
and
iv) at least one propellant.

Keratinic fibers as used herein are to be understood as furs, wool, feathers and, in particular, human hair. According to the above formulae and all of the following formulae, a chemical bond labeled with the symbol * denotes a free valency of the corresponding structural fragment.

Polymers as used herein are understood to be compounds that are different from the compounds of formulae (I) and (II) and possess a molecular weight of at least 10000 g/mol. The polymers are built up from a plurality of molecules, in which one or more types of atoms or atom groupings (so-called constitutive units, building blocks or repeating units) are repeatedly connected to one another. The polymers are obtained by polyreaction, the latter being able to take place artificially (i.e. synthetically) or naturally.

Fixing polymers contribute to the hold and/or to building up the hair volume and fullness of the overall style. At the same time, these polymers are also film-forming polymers and therefore are in general typical substances for hair-treatment agents having a shaping action, such as hair-fixing agents, hair mousses, hair waxes and hairsprays. It is quite possible for the film formation to be selective here, connecting only a few fibers together.

Film-forming polymers here are to be understood as those polymers that leave a continuous film on the skin, hair or nails upon drying. Film formers of this type can be used in a wide variety of cosmetic products, such as e.g. face masks, foundation, hair-fixing agents, hairsprays, hair gels, hair waxes, deep conditioners, shampoos or nail varnishes. Film-forming polymers are also understood as those polymers that are capable of depositing a transparent film on the hair when used in an about 0.01 to about 20 wt. % aqueous, alcoholic or aqueous-alcoholic solution.

Those cosmetic agents are preferred which contain as polymer at least one compound from the group of the nonionic polymers and cationic polymers.

Furthermore, those cosmetic agents that contain at least one fixing polymer, preferably at least one nonionic fixing polymer and/or at least one cationic fixing polymer, as polymer are most suitable. The polymers, in particular the fixing polymers, are contained preferably in a quantity of about 0.1 wt. % to about 15.0 wt. %, in particular of about 1.0 wt. % to about 12.0 wt. %, most particularly preferably of about 2.0 to about 10.0 wt. %, based in each case on the weight of the cosmetic agent.

Within the framework of a particularly preferred embodiment, the agents contain at least one nonionic fixing polymer as an additional fixing polymer. The additional nonionic fixing polymers are contained in the agent preferably in a quantity of about 0.1 wt. % to about 10.0 wt. %, particularly preferably of about 1.0 wt. % to about 8.0 wt. %, most particularly preferably of about 2.0 wt. % to about 6.0 wt. %, based in each case on the weight of the agent.

The nonionic fixing polymers are in turn preferably selected from at least one polymer from the group that is made up of
homopolymers and nonionic copolymers of N-vinylpyrrolidone,
nonionic copolymers of isobutene,
nonionic copolymers of maleic anhydride.
   Suitable polyvinylpyrrolidones are e.g. commercial products such as Luviskol® K 90 or Luviskol® K 85 from BASF SE. Suitable polyvinyl acetate is marketed, e.g., as an emulsion with the trade name Vinac® by Air Products. Agents that contain as nonionic fixing polymer at least one polymer selected from the group that is made up of copolymers of maleic anhydride and methyl vinyl ether,
polyvinylpyrrolidone,
copolymers of N-vinylpyrrolidone and vinyl esters of carboxylic acids with 2 to 18 carbon atoms, in particular of N-vinylpyrrolidone and vinyl acetate, or mixtures of these polymers, are most particularly preferred. In turn, those agents are preferred which contain as nonionic fixing polymer at least one polymer selected from the group that is made up of
polyvinylpyrrolidone,
copolymers of N-vinylpyrrolidone and vinyl esters of carboxylic acids with 2 to 18 carbon atoms, in particular N-vinylpyrrolidone and vinyl acetate,
or mixtures of these polymers.

If copolymers of N-vinylpyrrolidone and vinyl esters of carboxylic acids with 2 to 18 carbon atoms, in particular of N-vinylpyrrolidone and vinyl acetate, are employed, it is in turn preferred if the molar ratio of structural units obtained from the monomer N-vinylpyrrolidone to structural units obtained from the monomer vinyl esters of carboxylic acids with 2 to 18 carbon atoms (in particular vinyl acetate) in the polymer is in the range from about 20:80 to about 80:20 and in particular from about 30:70 to about 70:30. Suitable copolymers of vinylpyrrolidone and vinyl acetate are available, e.g., with the trademark Luviskol® VA 37, Luviskol® VA 55, Luviskol® VA 64 and Luviskol® VA 73 from BASF SE.

Cationic polymers are to be understood as polymers having a group that can be “temporarily” or “permanently” cationic in the main and/or side chain. “Permanently cationic” refers herein to those polymers that have a cationic group irrespective of the pH value of the agent. These are generally polymers that contain a quaternary nitrogen atom, for example in the form of an ammonium group. Preferred cationic groups are quaternary ammonium groups. In particular, those polymers in which the quaternary ammonium groups are bound via a C1-4 hydrocarbon group to a polymer main chain built up from acrylic acid, methacrylic acid or derivatives thereof have proved particularly suitable.

A cationic fixing polymer that is preferably suitable herein is at least one cationic fixing polymer containing at least one structural element of formula (M9) and additionally at least one structural element of formula (M10)

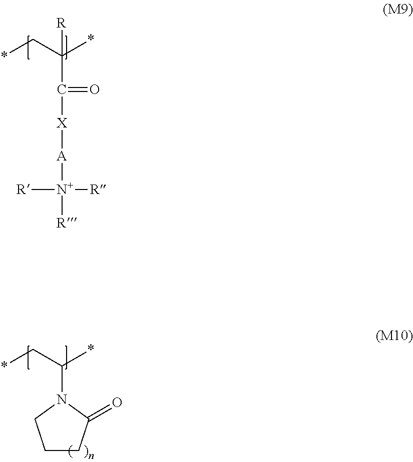

where
R denotes a hydrogen atom or a methyl group,
   R′, R″ and R′″ independently of one another denote a (C₁ to C₃₀) alkyl group,
X denotes an oxygen atom or an NH group,
A denotes an ethane-1,2-diyl group or a propane-1,3-diyl group,
n represents 1 or 3.

Also suitable as a particularly preferably suitable cationic fixing polymer is at least one amphiphilic, cationic polymer comprising at least one structural unit of formula (M6) and at least one structural unit of formula (VI) and optionally at least one structural unit of formula (V)

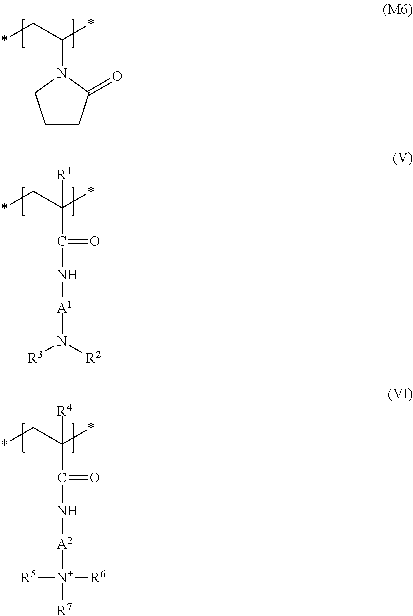

where
R¹ and R⁴ independently of one another denote a hydrogen atom or a methyl group, A¹ and A² independently of one another denote an ethane-1,2-diyl, propane-1,3-diyl or butane-1,4-diyl group,
R², R³, R⁵ and R⁶ independently of one another denote a (C₁ to C₄) alkyl group,
R⁷ denotes a (C₈ to C₃₀) alkyl group.

It is preferred if an agent contains as the above suitable cationic fixing polymer at least one amphiphilic, cationic polymer comprising at least one structural unit of formula (M6), at least one structural unit of formula (M7), at least one structural unit of formula (III) and at least one structural unit of formula (IV),

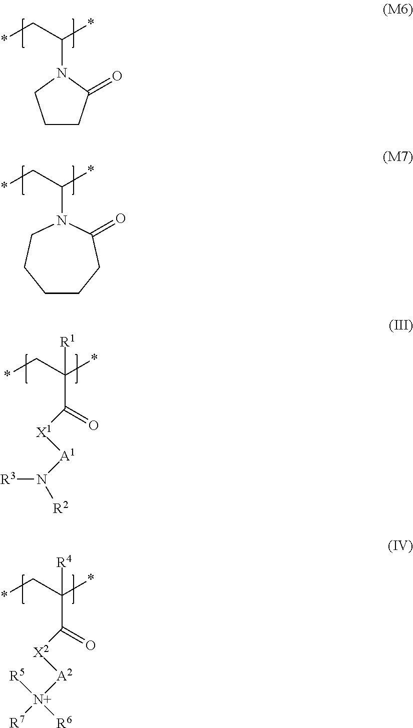

where
R¹ and R⁴ independently of one another denote a hydrogen atom or a methyl group,
X¹ and X² independently of one another denote an oxygen atom or an NH group,
A¹ and A² independently of one another denote an ethane-1,2-diyl, propane-1,3-diyl or butane-1,4-diyl group,
R², R³, R⁵ and R⁶ independently of one another denote a (C₁ to C₄) alkyl group,
R⁷ denotes a (C₈ to C₃₀) alkyl group.

To compensate for the positive polymer charge, all possible physiologically acceptable anions can be used, such as e.g. chloride, bromide, hydrogen sulfate, methyl sulfate, ethyl sulfate, tetrafluoroborate, phosphate, hydrogen phosphate, dihydrogen phosphate or p-toluenesulfonate, triflate.

These compounds are available for example as
copolymers of dimethylaminoethyl methacrylate, quaternized with diethyl sulfate, with vinylpyrrolidone (having the INCI name polyquaternium-11) with the names Gafquat® 440, Gafquat®734, Gafquat®755 (all ISP) and Luviquat PQ 11 PN (BASF SE),
copolymers of N-vinylpyrrolidone, N-(3-dimethylaminopropyl) methacrylamide and 3-(methacryloylamino)propyl lauryl dimethylammonium chloride (INCI name: polyquaternium-55), which is marketed, e.g., by ISP with the trade name Styleze W 10 or W 20 (W 10: 10 wt. % active substance and W 20: 20 wt. % active substance respectively, each in an ethanol-water mixture),
copolymers of N-vinylpyrrolidone, N-vinylcaprolactam, N-(3-dimethylaminopropyl) methacrylamide and 3-(methacryloylamino)propyl lauryl dimethylammonium chloride (INCI name: polyquaternium-69), which is marketed, e.g., by ISP with the trade name AquaStyle® 300 (28-32 wt. % active substance in an ethanol-water mixture).

Furthermore, the cationic film-forming and/or cationic fixing polymers are particularly preferably selected from cationic, quaternized cellulose derivatives.

Also suitable as a film-forming and/or fixing polymer are preferably cationic, quaternized cellulose derivatives. Those cationic, quaternized celluloses that carry more than one permanent cationic charge in a side chain have proved particularly advantageous herein. Among these cationic celluloses, in turn, those cationic celluloses with the INCI name polyquaternium-4, which are marketed, e.g., by National Starch with the names Celquat® H 100 and Celquat® L 200, are particularly suitable.

Furthermore, cationic polymers that can be used particularly preferably herein are those cationic fixing copolymers having at least one structural element of formula (M11)

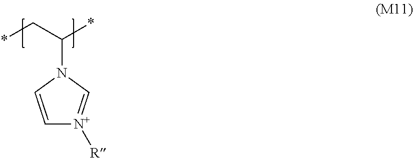

where R″ denotes a (C₁ to C₄) alkyl group, in particular a methyl group, and additionally at least one further cationic and/or nonionic structural element.

To compensate for the positive polymer charge, all possible physiologically acceptable anions can be used, such as e.g. chloride, bromide, hydrogen sulfate, methyl sulfate, ethyl sulfate, tetrafluoroborate, phosphate, hydrogen phosphate, dihydrogen phosphate or p-toluenesulfonate, triflate.

It is in turn preferred if, as an additional cationic fixing polymer, at least one copolymer (c1) is contained which, in addition to at least one structural element of formula (M11), also comprises a structural element of formula (M6)

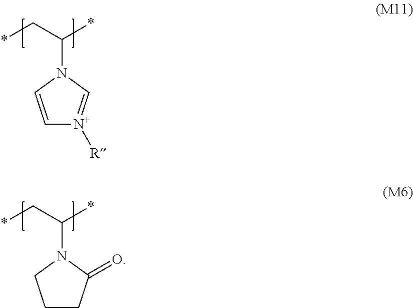

where R″ denotes a (C₁ to C₄) alkyl group, in particular a methyl group.

To compensate for the positive polymer charge of the copolymers (c1), all possible physiologically acceptable anions can be used, such as e.g. chloride, bromide, hydrogen sulfate, methyl sulfate, ethyl sulfate, tetrafluoroborate, phosphate, hydrogen phosphate, dihydrogen phosphate or p-toluenesulfonate, triflate.

Most particularly preferred cationic fixing polymers contain as copolymers (c1) about 10 to about 30 mole %, preferably about 15 to about 25 mole % and in particular about 20 mole % of structural units according to formula (M11) and about 70 to about 90 mole %, preferably about 75 to about 85 mole % and in particular about 80 mole % of structural units according to formula (M6).

It is particularly preferred here if the copolymers (c l) contain, in addition to polymer units resulting from the incorporation of the said structural units according to formula (M11) and (M6) into the copolymer, no more than about 5 wt. %, preferably no more than about 1 wt. %, of polymer units originating from the incorporation of other monomers. The copolymers (c1) are preferably built up exclusively from structural units of formula (M11) with R″=methyl and (M6). If a chloride ion is used to compensate for the positive charge of the polymer of the formula (poly1), these N-methylvinylimidazole/vinylpyrrolidone copolymers are referred to under INCI nomenclature as polyquaternium-16 and are available e.g. from BASF with the trade names Luviquat® Style, Luviquat® FC 370, Luviquat® FC 550, Luviquat® FC 905 and Luviquat® HM 552. If a methosulfate is used to compensate for the positive charge of the polymer of the formula (poly1), these N-methylvinylimidazole/vinylpyrrolidone copolymers are referred to under INCI nomenclature as polyquaternium-44 and are available e.g. from BASF with the trade name Luviquat® Ultracare.

In addition to or instead of the copolymer(s) (c1), the powdered compositions herein can also contain copolymers (c2), which, starting from the copolymer (c1), have as additional structural units structural units of formula (M7)

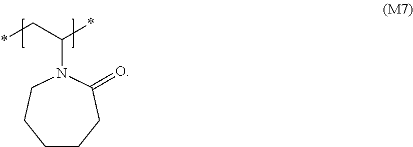

Other particularly preferred powdered compositions herein are therefore characterized in that they contain as cationic fixing polymer at least one copolymer (c2), which contains at least one structural unit according to formula (M11-a) and at least one structural unit according to formula (M6) and at least one structural unit according to formula (M7)

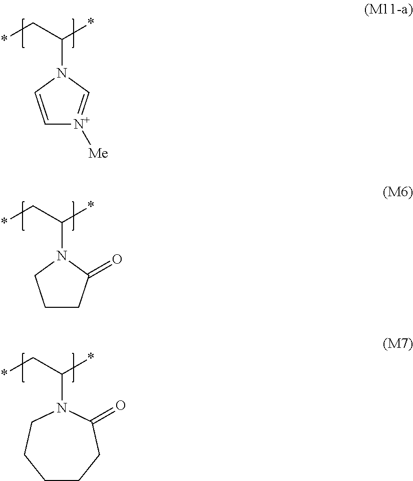

Here too, it is particularly preferred if the copolymers (c2) contain, in addition to polymer units resulting from the incorporation of the said structural units according to formula (M11-a), (M6) and (M7) into the copolymer, no more than about 5 wt. %, preferably no more than about 1 wt. %, of polymer units originating from the incorporation of other monomers. The copolymers (c2) are preferably built up exclusively from structural units of formulae (M11-a), (M6) and (M7).

To compensate for the positive polymer charge of component (c2), all possible physiologically acceptable anions can be used, such as e.g. chloride, bromide, hydrogen sulfate, methyl sulfate, ethyl sulfate, tetrafluoroborate, phosphate, hydrogen phosphate, dihydrogen phosphate or p-toluenesulfonate, triflate. If a methosulfate is used to compensate for the positive charge of the polymer of the formula (poly2), those N-methylvinyl-imidazole/vinylpyrrolidone/vinylcaprolactam copolymers are referred to under INCI nomenclature as polyquaternium-46 and are available e.g. from BASF with the trade name Luviquat® Hold.

Most particularly preferred copolymers (c2) contain about 1 to about 20 mole %, preferably about 5 to about 15 mole % and in particular about 10 mole % of structural units according to formula (M11-a) and about 30 to about 50 mole %, preferably about 35 to about 45 mole % and in particular about 40 mole % of structural units according to formula (M6) and about 40 to about 60 mole %, preferably about 45 to about 55 mole % and in particular about 60 mole % of structural units according to formula (M7).

In addition to or instead of the copolymer(s) (c1) and/or (c2), the powdered compositions can also contain, as cationic fixing polymer, copolymers (c3), which have structural units of formulae (M11-a) and (M6) as structural units as well as further structural units from the group of the vinylimidazole units and further structural units from the group of the acrylamide and/or methacrylamide units.

Further particularly preferred agents herein are characterized in that they contain as an additional cationic film-forming and/or cationic fixing polymer at least one copolymer (c1), which contains at least one structural unit according to formula (M11-a) and at least one structural unit according to formula (M6) and at least one structural unit according to formula (M10) and at least one structural unit according to formula (M12)

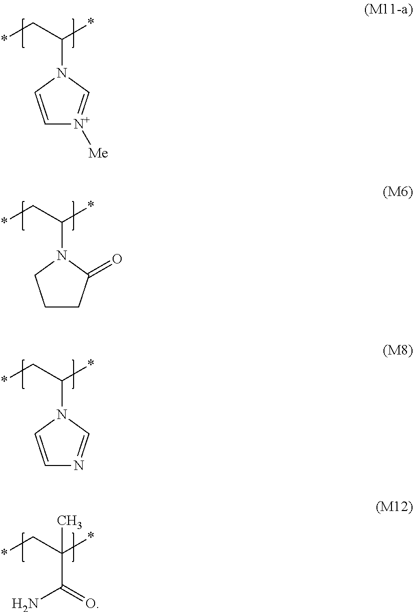

Here too, it is particularly preferred if the copolymers (c1) contain, in addition to polymer units resulting from the incorporation of the said structural units according to formulae (M11-a), (M6), (M8) and (M12) into the copolymer, no more than about 5 wt. %, preferably no more than about 1 wt. %, of polymer units originating from the incorporation of other monomers. The copolymers (c1) are preferably built up exclusively from structural units of formulae (M11-a), (M6), (M8) and (M12).

To compensate for the positive polymer charge of component (c1), all possible physiologically acceptable anions can be used, such as e.g. chloride, bromide, hydrogen sulfate, methyl sulfate, ethyl sulfate, tetrafluoroborate, phosphate, hydrogen phosphate, dihydrogen phosphate or p-toluenesulfonate, triflate.

If a methosulfate is used to compensate for the positive charge of the polymer of the formula (poly3), those N-methylvinylimidazole/vinyl-pyrrolidone/vinylimidazole/methacrylamide copolymers are referred to under INCI nomenclature as polyquaternium-68 and are available, e.g., from BASF with the trade name Luviquat® Supreme.

Further cationic fixing polymers that can preferably be used in the agents contemplated herein are the so-called “temporarily cationic” polymers. These polymers generally contain an amino group which is present at certain pH values as a quaternary ammonium group and therefore in cationic form.

As temporarily cationic polymers, chitosans represent cationic fixing polymers. From a chemical point of view, chitosans are partially deacetylated chitins of different molecular weights.

Chitosan is produced starting from chitin, preferably from the shell residues of crustaceans, which are available in large quantities as inexpensive raw materials. Chitin is generally first deprotonated by adding bases, demineralized by adding mineral acids and finally deacetylated by adding strong bases, in which case the molecular weights can be distributed over a broad range. Those grades having an average molecular weight (weight average) of 800,000 to 1,200,000 daltons, a Brookfield viscosity (1 wt. % in glycolic acid) below 5000 mPas, a degree of deacetylation in the range of 80 to 88% and an ash content of less than 0.3 wt. % are preferably employed.

In addition to the chitosans as typical biopolymers, cationically derivatized chitosans (such as e.g. quaternization products) or alkoxylated chitosans are also suitable as derivatives of chitosan.

In an exemplary embodiment, the agents are characterized in that they contain as a cationic fixing polymer at least one neutralization product of chitosan with at least one organic carboxylic acid, such as in particular formic acid, acetic acid, citric acid, lactic acid, pyrrolidone carboxylic acid, tartaric acid, glycolic acid, nicotinic acid, hydroxyisobutyric acid, hydroxyisovaleric acid or mixtures of these acids. It is preferred to select the organic carboxylic acid from lactic acid, formic acid, pyrrolidone carboxylic acid, nicotinic acid, hydroxyisobutyric acid, hydroxyisovaleric acid or mixtures of these acids. This neutralization product can be produced for example in an aqueous medium by addition of chitosan and the appropriate organic carboxylic acid.

Suitable chitosans are readily available commercially, e.g., with the trade names Hydagen® CMF (1 wt. % active substance in aqueous solution with 0.4 wt. % glycolic acid, molecular weight 500000 to 5000000 g/mol Cognis), Hydagen® HCMF (chitosan (80% deacetylated), molecular weight 50000 to 1000000 g/mol, Cognis), Kytamer® PC (80 wt. % active substance of chitosan pyrrolidone carboxylate (INCI name: chitosan PCA), Amerchol) and Chitolam® NB/101.

Chitosans or derivatives thereof are contained in the agents contemplated herein preferably in a quantity of about 0.01 wt. % to about 5.0 wt. %, particularly preferably of about 0.05 wt. % to about 2.0 wt. %, most particularly preferably of about 0.1 wt. % to about 1.0 wt. %, based in each case on the weight of the agent.

Preferably suitable temporarily cationic polymers are also considered to be those that are not permanently cationic and have at least one structural unit of formulae (M1-1) to (M1-8)

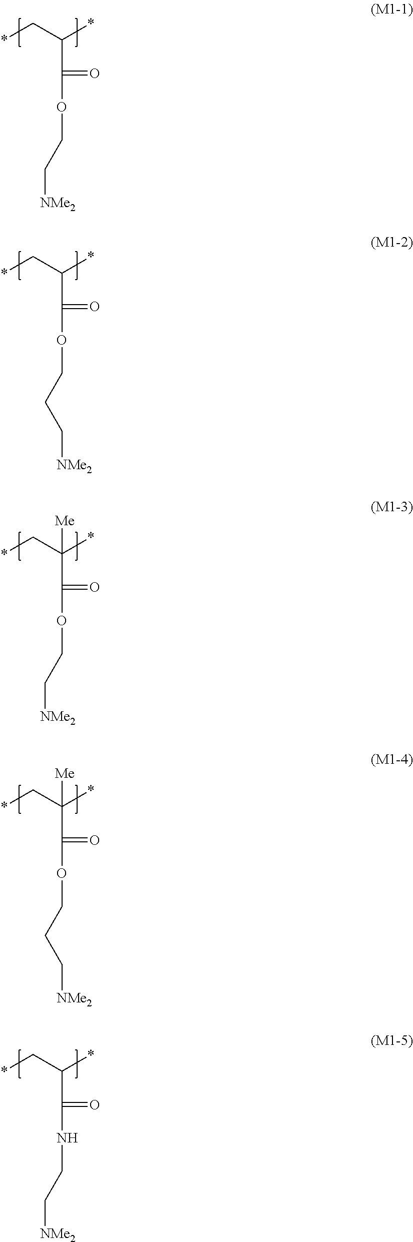

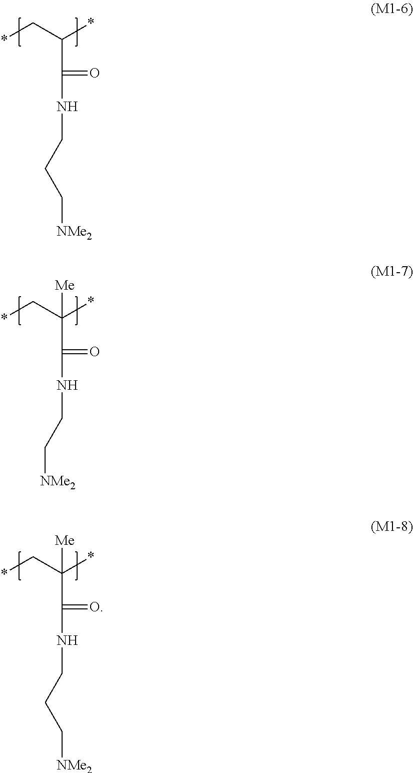

Here, those copolymers are in turn preferred which contain at least one structural unit of formulae (M1-1) to (M1-8) and additionally at least one structural unit of formula (M10),

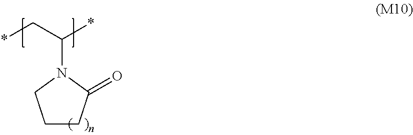

where
n represents 1 or 3.

Here, the group of polymers
vinylcaprolactam/vinylpyrrolidone/dimethylaminoethyl methacrylate copolymer (e.g. INCI name: vinyl caprolactam/PVP/dimethylaminoethyl methacrylate copolymer with the trade name Gaffix® VC 713 (ISP)),
vinylpyrrolidone/vinylcaprolactam/dimethylaminopropyl methacrylamide copolymer (e.g. INCI name: VP/vinyl caprolactam/DMAPA acrylates copolymer with the trade name Aquaflex® SF-40 (ISP)),
vinylcaprolactam/vinylpyrrolidone/dimethylaminoethyl methacrylate copolymer (e.g. as 35-39% solids in ethanol in the form of the commercial product Advantage LC E with the INCI name: vinyl caprolactam/VP/dimethylaminoethyl methacrylate copolymer, alcohol, lauryl pyrrolidone (ISP)),
vinylpyrrolidone/dimethylaminopropyl methacrylamide copolymer (e.g. INCI name:

VP/DMAPA acrylates copolymer with the trade name Styleze CC-10 (ISP)), is in turn considered to be a preferred list for the selection of at least one or more polymers therefrom.

Preferred cosmetic agents contain those compounds of formula (I) in which X¹ denotes a direct bond.

Moreover, it has proved to be preferred if the compounds of formula (I) are selected from those in which n according to formula (I) denotes an integer from 1 to 10.

The effect can be further optimized by the compounds of formula (I) contained in the agent contemplated herein possessing a molecular weight of no more than about 1200 g/mol, in particular no more than about 750 g/mol.

Preferred compounds of formula (I) are selected from among at least one compound from the group that is made up of oleyl alcohol with 2 mole equivalents of ethylene oxide (oleth-2), oleyl alcohol with 5 mole equivalents of ethylene oxide (oleth-5), oleyl alcohol with 10 mole equivalents of ethylene oxide (oleth-10), stearyl alcohol with 10 equivalents of ethylene oxide (steareth-10), cetearyl alcohol with 6 mole equivalents of ethylene oxide (ceteareth-6), cetearyl alcohol with 12 mole equivalents of ethylene oxide (ceteareth-12), lauryl alcohol with 2 equivalents of ethylene oxide (laureth-2), lauryl alcohol with 3 equivalents of ethylene oxide (laureth-3), lauryl alcohol with 4 equivalents of ethylene oxide (laureth-4), lauryl alcohol with 10 equivalents of ethylene oxide (laureth-10), and caprylic alcohol with 4 equivalents of ethylene oxide (capryleth-4).

Based on the total weight of the agents contemplated herein, the compounds of formula (I) are preferably contained in a quantity of about 0.05 to about 0.4 wt. %.

Furthermore, it has been shown that the compounds of formula (II) contained in the agent preferably possess a molecular weight of at least about 1450 g/mol, in particular at least about 1550 g/mol.

It is preferred if the compounds according to formula (II) are selected from at least one compound of formula (II-a)

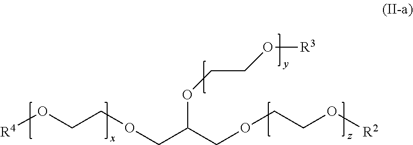

where
R², R³ and R⁴ independently of one another denote a hydrogen atom, a linear or branched (C₇ to C₂₁) alkylcarbonyl group or a linear or branched (C₇ to C₂₁) alkenylcarbonyl group, x, y and z independently of one another denote an integer from 0 to 80, with the provisos that
at least one group from R², R³, R⁴ is different from a hydrogen atom and
the sum of x+y+z is at least 30.

Preferred representatives of these compounds of formula (II-a) are hydrogenated triglycerides containing 30 to 80 mole equivalents of ethylene oxide units. These preferred hydrogenated and ethoxylated triglycerides are particularly preferably selected from at least one compound from the group that is made up of PEG-40 hydrogenated castor oil, PEG-50 hydrogenated castor oil, PEG-54 hydrogenated castor oil, PEG-60 hydrogenated castor oil, PEG-40 castor oil, PEG-50 castor oil, PEG-54 castor oil and PEG-60 castor oil.

Based on the total weight of the agents contemplated herein, the compounds of formula (II) are preferably contained in a quantity of about 0.05 to about 0.4 wt. %.

Preferred cosmetic agents are characterized in that the quantity of the compounds of formula (I) and of the compounds of formula (II) is in a weight ratio range from about 4:1 to about 1:4, in particular from about 3:1 to about 1:3.

A preferred cosmetic agent for the cosmetic treatment of keratinic fibers, in particular human hair, contains in a cosmetic carrier, based on the weight of the total agent,
i) about 0.1 wt. % to about 15.0 wt. %, in particular about 1.0 wt. % to about 12.0 wt. %, most particularly preferably about 2.0 to about 10.0 wt. %, of at least one fixing polymer and
ii) about 0.05 to about 0.4 wt. % of at least one compound of formula (I) comprising no more than 20 ethylene oxide units,

R¹—X¹—(O—CH₂CH₂)ₙ—OH (I)
where
R¹ denotes a linear or branched, saturated or unsaturated hydrocarbon residue with 8 to 20 carbon atoms,
X¹ denotes a direct bond or a carbonyl group,
n denotes an integer from 1 to 20,
   and
iii) about 0.05 to about 0.4 wt. % of at least one linear or branched compound of formula (II) with at least 30 ethylene oxide units

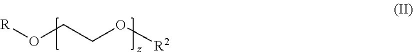

where
R² denotes a linear or branched (C₇ to C₂₁) alkylcarbonyl group, a linear or branched (C₇ to C₂₁) alkenylcarbonyl group or denotes a linear or branched, saturated or unsaturated (C₈ to C₂₂) hydrocarbon residue,
R denotes a hydrogen atom or a group of formula (III)

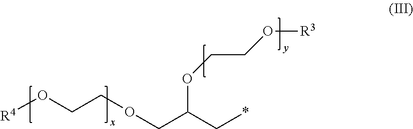

in which x and y independently of one another denote an integer from 0 to 80,
R³ and R⁴ independently of one another denote a hydrogen atom or a linear or branched, saturated or unsaturated (C₇ to C₂₁) alkylcarbonyl group,

z denotes an integer from 0 to 80,

with the provisos that
if R is a group of formula (III), at least one group from R², R³, R⁴ denotes a linear or branched (C₇ to C₂₁) alkylcarbonyl group or a linear or branched (C₇ to C₂₁) alkenylcarbonyl group and the sum of x+y+z is at least 30,
if R denotes a hydrogen atom, z denotes an integer from 30 to 80
   and
iv) about 1.0 to about 35.0 wt. % of at least one propellant (in particular propane and/or butane).

The agents contemplated herein contain their active agents in a cosmetic carrier, preferably in an aqueous cosmetic carrier, alcoholic cosmetic carrier or an aqueous-alcoholic cosmetic carrier. It is preferred if it is an aqueous cosmetic carrier or an aqueous-alcoholic cosmetic carrier. It is preferred if the cosmetic carrier of the agent contains water. Particularly preferably, the agent contains at least about 50 wt. % water, based on the weight of the total agent.

Aqueous-alcoholic carriers as used herein are to be understood as aqueous compositions containing about 3 to about 70 wt. % of a C₁-C₄ alcohol, in particular ethanol or isopropanol. The agents can additionally contain further organic solvents, such as e.g. methoxybutanol, benzyl alcohol, ethyl diglycol, 1,2-propylene glycol or 1,3-propylene glycol. All water-soluble organic solvents are preferred here.

Furthermore, the agent contemplated herein can additionally contain at least one cationic surfactant to increase the haircare action. As contemplated herein, cationic surfactants are not understood as being polymers. Examples of the cationic surfactants that can be employed in the agents are, in particular, quaternary ammonium compounds with one or two (C₈ to C₂₀) alkyl residues. Preferred are ammonium halides, in particular chlorides and bromides, such as alkyltrimethylammonium chlorides, dialkyldimethylammonium chlorides and trialkylmethylammonium chlorides, e.g. cetyltrimethylammonium chloride, stearyltrimethylammonium chloride, distearyldimethylammonium chloride, lauryldimethylammonium chloride, lauryldimethylbenzylammonium chloride and tricetylmethylammonium chloride. Furthermore, the very readily biodegradable quaternary ester compounds, so-called “esterquats”, such as e.g. the methyl hydroxyalkyldialkoyloxyalkylammonium methosulfates marketed with the trademarks Dehyquart® and Stepantex®, can be employed as said quaternary ammonium compounds.

It has proved to be particularly preferred if the agents contemplated herein are free from quaternary ammonium compounds having one or two (C₈ to C₂₀) alkyl residues.

The agents herein are preferably present as a foam. To this end, the agents are packed in a compressed gas container with a delivery device (“aerosol container”). The compressed gas container, with the aid of which a product is distributed by the internal gas pressure of the container through a valve, is referred to by definition as an “aerosol container”. Conversely to the definition of an aerosol, a vessel under normal pressure is defined as a “non-aerosol container”, with the aid of which a product is distributed by means of mechanical action by a pumping or squeezing system.

In general, all constituents of the agent contemplated herein, with the exception of the propellant, are packed in a suitable pressure-resistant container. This is then sealed with a valve. Using conventional techniques, the desired quantity of propellant is finally added.

In the embodiment as an aerosol foam, suitable propellants useful herein are selected e.g. from N₂O, dimethyl ether, CO₂, air, alkanes with 3 to 5 carbon atoms, such as propane, n-butane, isobutane, n-pentane and isopentane, and mixtures thereof. According to the embodiment of an aerosol foam, the said alkanes, mixtures of said alkanes or mixtures of said alkanes with dimethyl ether are employed as the only propellant. However, propellants of the type of the hydrochlorofluorocarbons, but in particular the hydrofluorocarbons, also can be used. Preferred are dimethyl ether, propane, n-butane, isobutane and mixtures thereof. Most particularly preferably, mixtures of propane and butane are used as the sole propellant in a weight ratio of propane to butane of about 70:30 to about 15:85. These mixtures in turn are preferably employed in the agents in a quantity of about 2.0 to about 15.0 wt. %, based on the weight of the total agent. Butane is understood herein to be n-butane, isobutane and mixtures of n-butane and isobutane.

When conventional aerosol containers are used, the agents contemplated herein contain the propellant preferably in quantities of about 1 to about 35 wt. %, based on the total agent. Quantities of about 2 to about 30 wt. %, in particular of about 3 to about 15 wt. %, are particularly preferred.

For the foaming of agents in gel form in a dual-chamber aerosol container, isopentane is preferably suitable as a propellant, which is incorporated into the agents and packed in the first chamber of the dual-chamber aerosol container. In the second chamber of the dual-chamber aerosol container, at least one further propellant other than isopentane is packed, which builds up a higher pressure than isopentane in the dual-chamber aerosol container. The propellants in the second chamber are preferably selected from N₂O, dimethyl ether, CO₂, air, alkanes with 3 or 4 carbon atoms (such as propane, n-butane, isobutane) and mixtures thereof.

In another exemplary embodiment, the use of a cosmetic agent described above is provided for the temporary shaping and/or shape-fixing of keratinic fibers, in particular human hair.

In a further exemplary embodiment, a method for the temporary shaping of keratinic fibers, in particular human hair, is characterized in that a cosmetic agent described above is applied onto the keratinic fibers.

It has proved to be preferred if the keratinic fibers, after exposure to the cosmetic agents contemplated herein are not rinsed and the cosmetic agents are left on the fiber.

The following are examples of agents contemplated herein. The examples are provided for illustration purposes only and are not meant to limit the various embodiments in any way.

### EXAMPLES

All quantitative data, unless otherwise shown, are expressed in weight percent. The following formulations were produced as an aerosol foam product:

| Raw material | E1 | E2 | E3 | E4 | E5 | V1 | V2 |
|---|---|---|---|---|---|---|---|
| Luviskol ® 60/40 | 5.00 | 5.00 | 5.00 | 5.00 | 5.00 | 5.00 | 5.00 |
| W NP¹ | | | | | | | |
| Styleze ® W 10² | 2.00 | 2.00 | 2.00 | 2.00 | 2.00 | 2.00 | 2.00 |
| Polyquaternium- | 1.00 | 1.00 | 1.00 | 1.00 | 1.00 | 1.00 | 1.00 |
| 16 | | | | | | | |
| PEG-40 | 0.20 | 0.20 | 0.20 | — | — | 0.40 | — |
| Hydrogenated | | | | | | | |
| Castor Oil | | | | | | | |
| Eumulgin ® B3³ | — | — | — | 0.20 | 0.20 | | |
| Eumulgin ® B1⁴ | 0.20 | — | 0.10 | — | 0.10 | — | 0.10 |
| Eumulgin ® O5⁴ | — | 0.20 | 0.10 | 0.20 | 0.10 | | 0.10 |

| | |
|---|---|
| Water | to 100 |

¹copolymer of N-vinylpyrrolidone and vinyl acetate
²copolymer of N-vinylpyrrolidone, N,N-dimethylaminopropyl methacrylamide and N,N-dimethyl-N-dodecylammoniopropyl methacrylamide chloride (10 wt. % active substance, INCI name: polyquaternium-55) (ISP)
³cetyl stearyl alcohol with approx. 30 EO units (INCI name: ceteareth-30) (Cognis)
⁴cetyl stearyl alcohol with approx. 12 EO units (INCI name: ceteareth-12) (Cognis)
⁵oleyl cetyl alcohol with approx. 5 EO units, plant-based (INCI name: oleth-5) (Cognis)

92 g of the agents E1 to E5 formed as described herein and of the comparative formulations V1 and V2 were each packed into a pressure vessel. The pressure vessels were sealed with a valve and 8 g of a propellant (propane/butane 15/85) was added. The agents were each delivered as a foam. The agents displayed a foam that formed rapidly and was stable on the inner surface of the hand. The foam of comparative formulation V1 formed slowly. The comparative formulation V2 yielded a foam that broke down rapidly on the inner surface of the hand and thus was more difficult to use.

Each foam was applied onto a moistened strand of hair for shape fixing, and wound on a spiral roller. The hair that had been treated with the agents contemplated herein retained a natural gloss, strong hold and lasting volume. Hair that had been treated with the foam of comparative formulation V1 was clearly weighed down and heavy and retained the shape less well.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

1. A cosmetic agent for the cosmetic treatment of keratinic fibers comprising in a cosmetic carrier:
i) at least one polymer;
ii) at least one compound of formula (I) comprising no more than 20 ethylene oxide units
R¹—X¹—(O—CH₂CH₂)ₙ—OH (I)
where
R¹ denotes a linear or branched, saturated or unsaturated hydrocarbon residue with 8 to 20 carbon atoms,
X¹ denotes a direct bond or a carbonyl group,
n denotes an integer from 1 to 20;
iii) at least one linear or branched compound of formula (II) with at least 30 ethylene oxide units 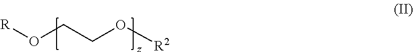
where
R² denotes a linear or branched (C₇ to C₂₁) alkylcarbonyl group, a linear or branched (C₇ to C₂₁) alkenylcarbonyl group or denotes a linear or branched, saturated or unsaturated (C₈ to C₂₂) hydrocarbon residue,
R denotes a hydrogen atom or a group of formula (III) 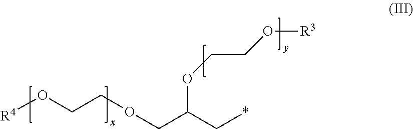
in which x and y independently of one another denote an integer from 0 to 80,
R³ and R⁴ independently of one another denote a hydrogen atom or a linear or branched, saturated or unsaturated (C₇ to C₂₁) alkylcarbonyl group,
z denotes an integer from 0 to 80,
with the provisos that
if R is a group of formula (III), at least one group from R², R³, R⁴ denotes a linear or branched (C₇ to C₂₁) alkylcarbonyl group or a linear or branched (C₇ to C₂₁) alkenylcarbonyl group and the sum of x+y+z is at least 30,
if R denotes a hydrogen atom, z signifies an integer from 30 to 80,
and
iv) at least one propellant.

2. The cosmetic agent according to claim 1, wherein the at least one polymer is chosen from the group comprising nonionic polymers and cationic polymers.

3. The cosmetic agent according to claim 1, wherein the at least one polymer is a fixing polymer.

4. The cosmetic agent according to claim 1, wherein the polymers are contained in the cosmetic agent in a quantity of about 0.1 wt. % to about 15.0 wt. %, based in each case on the weight of the cosmetic agent.

5. The cosmetic agent according to claim 1, wherein according to formula (I), X¹ denotes a direct bond.

6. The cosmetic agent according to claim 1, wherein according to formula (I), n denotes an integer from 1 to 10.

7. The cosmetic agent according to claim 1, wherein the at least one compounds of formula (I) has a molecular weight of no more than about 1200 g/mol.

8. The cosmetic agent according to claim 1, wherein the at least one linear or branched compound of formula (II) is selected from at least one compound of formula (II a), 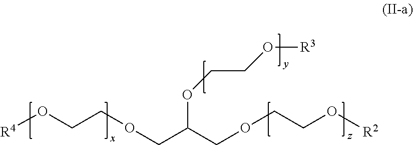
where
R², R³ and R⁴ independently of one another denote a hydrogen atom, a linear or branched (C₇ to C₂₁) alkylcarbonyl group or a linear or branched (C₇ to C₂₁) alkenylcarbonyl group,
x, y and z independently of one another denote an integer from 0 to 80,
with the provisos that
at least one group from R², R³, R⁴ is different from a hydrogen atom and
the sum of x+y+z is at least 30.

9. The cosmetic agent according to claim 8, wherein according to formula (II-a) R², R³ and R⁴ independently of one another denote a linear or branched, saturated or unsaturated (C₇ to C₂₁) alkylcarbonyl group.

10. The cosmetic agent according to claim 1, wherein the quantity of the at least one compounds of formula (I) and of the at least one linear or branched compounds of formula (II) are in a weight ratio range of about 4:1 to about 1:4.

11. The cosmetic agent according to claim 1, further comprising a cosmetic carrier comprising water.

12. The cosmetic agent according to claim 1, wherein the cosmetic agent is adapted to be dispensed as a foam.

13. The cosmetic agent according to claim 3, wherein the at least one fixing polymer is a nonionic fixing polymer and/or a cationic fixing polymer.

14. The cosmetic agent according to claim 4, wherein the polymers are contained in the cosmetic agent in a quantity of about 1.0 wt. % to about 12.0 wt. %, based on the weight of the cosmetic agent.

15. The cosmetic agent according to claim 4, wherein the polymers are contained in the cosmetic agent in a quantity of about 2.0 wt. % to about 10.0 wt. %, based on the weight of the cosmetic agent.

16. The cosmetic agent according to claim 10, wherein the quantity of the at least one compound of formula (I) and of the at least one linear or branched compound of formula (II) are in a weight ratio range of about 3:1 to about 1:3.
